(19) United States
(12) Patent Application Publication
(10) Pub. No.: US 2022/0211088 A1
(43) Pub. Date: Jul. 7, 2022

(54) SCUTELLARIA COMPOSITIONS AND METHODS FOR TASTE MODULATION

(71) Applicant: INTERNATIONAL FLAVORS & FRAGRANCES INC., Union Beach, NJ (US)

(72) Inventors: Hou WU, Union Beach, NJ (US); Diana Klaser CHENG, Union Beach, NJ (US); Jung-A KIM, Union Beach, NJ (US); Thumpalasseril V JOHN, Union Beach, NJ (US)

(73) Assignee: INTERNATIONAL FLAVORS & FRAGRANCES INC., Union Beach, NJ (US)

(21) Appl. No.: 17/604,011

(22) PCT Filed: Mar. 30, 2020
(86) PCT No.: PCT/US2020/025698

§ 371 Date: Oct. 15, 2021

Related U.S. Application Data
(60) Provisional application No. 62/833,839, filed on Apr. 15, 2019.

(51) Int. Cl. A23L 27/00; A23L 27/10; A61K 8/9789; A61K 47/46; A61Q 11/00

(52) U.S. Cl. CPC A23L 27/84; A23L 27/86; A23L 27/10; A23V 2002/00; A61K 47/46; A61Q 11/00; A61K 8/9789

(57) ABSTRACT

A Scutellaria extract, as well as a carbohydrase-treated Scutellaria extract and flavones obtained from Scutellaria are described for use in compositions and methods for improving the taste of a consumable containing a component having an astringent, bitter or off-taste.

### INTRODUCTION

This application is a 371 of International Application Serial No. PCT/US2020/025698, filed Mar. 30, 2020 and claims the benefit of priority from U.S. Patent Application Ser. No. 62/833,839, filed Apr. 15, 2019, the content of which is incorporated herein by reference in its entirety.

### BACKGROUND

The genus Scutellaria contain over 350 species represented by perennial and annual herbs, some of which are widely used in traditional medicine especially in China, Korea, and Japan due to their anti-inflammatory, antiviral, sedative, antithrombotic, and antioxidant effects. These effects are correlated with the content of flavonoids, among which, baicalin, baicalein, and wogonoside are the major compounds. Baicalein and baicalin exhibit superior free radical scavenging and have been shown to attenuate oxidative stress in cardiomyocytes and neuronal cells. In addition, wogonoside has strong activity against lipid peroxidation and an inhibitory effect on histamine and IgE production. In this respect, CN 100423736 C suggests the use of baicalin in combination with amantadine hydrochloride in a composition for use in the treatment of influenza.

KR 101169587 B1 suggests improving the taste of a Scutellaria baicalensis extract by fermenting the extract with a lactic acid bacterium thereby reducing the bitter taste of the extract.

U.S. Pat. No. 8,435,586 B2 discloses a method for intensifying a sensory impression of alcohol by adding to the alcohol a 2-phenyl-chromen-4-one.

CN 10480054 A discloses a medicine for treating bitter taste, which includes Prunella vulgaris, honeysuckle, Dendranthema morifolium, Chinese wolfberry, Radix Rehmanniae, Radix Ophiopogonis, Radix Gentianae, Gardenia jasminoides, Scutellaria baicalensis, and Radix Bupleuri.

### SUMMARY OF THE INVENTION

This invention provides a consumable including a component having an astringent, bitter or off-taste; and a Scutellaria extract or one or more flavones thereof. The invention also provides a method for improving the taste of a consumable by adding to a consumable having a component with an astringent, bitter or off-taste, a Scutellaria extract or one or more flavones thereof in an amount effective to reduce or suppress said astringent, bitter or off-taste. A component having an astringent, bitter or off-taste can include a protein, carbohydrate sweetener, artificial sweetener or preservative. In some aspects, the Scutellaria extract is a carbohydrate-treated Scutellaria extract or Scutellaria baicalensis extract. In other aspects, the one or more flavones include flavone aglycones (e.g., baicalein, wogonin, oroxylin A or a combination thereof) and/or flavone glycosides (baicalin, wogonoside, oroxylin A glucuronide or a combination thereof). In certain embodiments, the Scutellaria extract or one or more flavones thereof is present in an amount of 0.01 ppm or greater; in an amount in the range of 0.05 ppm to 500 ppm; in an amount in the range of 0.1 ppm to 100 ppm; or in an amount in the range of 0.5 ppm to 50 ppm. In a further aspect, the consumable is a food product, pharmaceutical composition, a dietary supplement, a nutraceutical, a dental hygienic composition, a tabletop sweetener, a beverage, or a cosmetic product.

### DETAILED DESCRIPTION OF THE INVENTION

It has now been found that an extract of Scutellaria, as well as a carbohydrate-treated extract thereof and flavones isolated from the same, effectively mask the bitter, astringent and off-tastes of consumable products. In particular, it has been shown that flavone aglycones and flavone glycosides of a S. baicalensis extract reduce or suppress bitter, astringent and off-tastes associated with proteins, carbohydrate sweeteners, artificial sweeteners, and/or preservatives such as benzoic acid or sorbic acid. Accordingly, the present invention provides consumables and methods, which include a Scutellaria extract and/or one or more flavones isolated from the Scutellaria extract as additives to improve the taste of the consumable by reducing or suppressing the astringency, bitterness and/or off-taste of the consumable.

As used herein, a Scutellaria extract is an extract from the roots or aerial part of a plant in the genus Scutellaria. In some embodiments, the plant in the genus Scutellaria is S. baicalensis, S. lateriflora, S. racemosa, S. ocellate, S. alpine, S. galericulata, S. tomentosa, S. wrightii, S. barbata, S. litwinowii, S. amoena, S. prostrata, S. rivularis, S. discolor, S. ramosissima, S. havanensis, or S. supina. In certain embodiments, the plant in the genus Scutellaria is S. baicalensis (also known as Skullcap). In other embodiments, the Scutellaria extract is an extract from the root of the plant. In particular embodiments, the Scutellaria extract of the invention is an extract of the root of S. baicalensis or S. lateriflora.

Preferably, the Scutellaria extract is enriched for flavones, in particular flavone glycosides such as Baicalin (5,6-dihydroxy-7-O-glucuronide flavone; CAS No. 21967-41-9), Wogonoside (wogonin 7-O-β-D-glucuronide; CAS No. 51059-44-0), 5,7-dihydroxy-6-methoxyflavone-7-O-β-D-glucuronopyranoside (Oroxylin A glucuronide); and/or flavone aglycones such as baicalein (5,6,7-trihydroxyflavone or 5,6,7-Trihydroxy-2-phenyl-chromen-4-one; CAS No. 491-67-8), wogonin (5,7-dihydroxy-8-methoxyflavone or 5,7-dihydroxy-8-methoxy-2-phenyl-4H-chromen-4-one; CAS No. 632-85-9) and/or 5,7-dihydroxy-6-methoxyflavone (oroxylin A) ((Table 1).

TABLE 1
Flavone Glycosides

| | | |
|---|---|---|
| 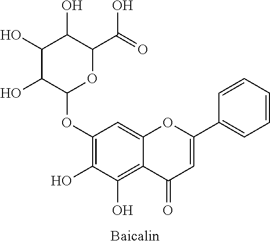 [SMILES: O=C(O)C1OC(Oc2cc3oc(-c4ccccc4)cc(=O)c3c(O)c2O)C(O)C(O)C1O] | 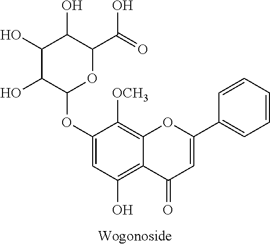 [SMILES: COc1c(OC2OC(C(=O)O)C(O)C(O)C2O)cc(O)c2c(=O)cc(-c3ccccc3)oc12] | 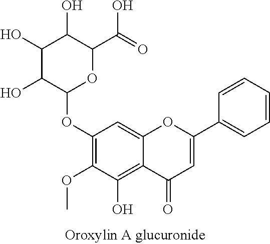 [SMILES: COc1c(OC2OC(C(=O)O)C(O)C(O)C2O)cc2oc(-c3ccccc3)cc(=O)c2c1O] |

Flavone Aglycones

| | | |
|---|---|---|
| 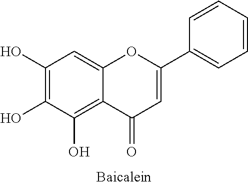 [SMILES: O=c1cc(-c2ccccc2)oc2cc(O)c(O)c(O)c12] | 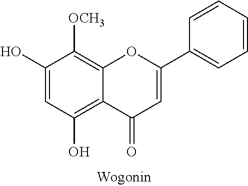 [SMILES: COc1c(O)cc(O)c2c(=O)cc(-c3ccccc3)oc12] | 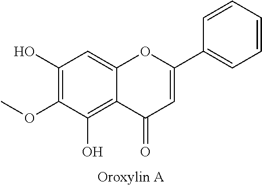 [SMILES: COc1c(O)cc2oc(-c3ccccc3)cc(=O)c2c1O] |

While plants in the genus Scutellaria have been shown to include one or more of baicalin, baicalein, wogonoside, wogonin (Zhao, et al. (2016) Sci. Bull. (Beijing) 61(18):1391-8; Gao, et al. (2008) J. Pharm. Pharm. Sci. 11(1):77-87; Cole, et al. (2008) Planta Med. 74(4):474-81; Kikuchi, et al. (1991) Chem. Pharm. Bull. 39(1):199-201; Kosakowska, et al. (2016) Herba Polonica 62(3):7-19; Islam, et al. (2010) Metabolomics 7(3):446-53; Tayarani-Najarani, et al. (2012) Braz. J. Pharmacog. 22(2):268-76; Lin & Shieh (1996) Am. J. Chin. Med. 24(1):31-6; Marrero, et al. (2015) Internatl. J. Pharmaceut. Sci. Rev. Res. 30(2):104-8) these compounds have also been reported in Oroxylum indicum (Indian trumpet flower) (Raghu, et al. (2013) J. Pharmacog Phytochem. 2(3):23-27; Majeed, et al. (2017) J. Liq. Chromatog. Rel. Technol. 40(14):732-40), baicalein has been found in Thymus vulgaris (Fujita, et al. (2005) Microbiol. Immunol. 49(4):391-6), and wogonoside has been isolated from Bacopa monnieri and Holmskioldia sanguinea (Chinese hat plant) (Chaudhuri, et al. (2004) Phytother. Res. 18(2):114-7). Accordingly, the flavones of this invention may also be obtained from one or more of these alternative sources.

A Scutellaria extract can be obtained by grinding, milling or pulverizing dried Scutellaria plant material (e.g., dried S. baicalensis root) to obtain a powder and subsequently suspending the powder in 50-75% ethanol (preferably about 70% ethanol) for a time sufficient to extract the desired flavones from the plant material (e.g., 30 minutes to 24 hours) and filtering the extract to remove insoluble plant material (Yu, et al. (2013) Oncol. Rep. 30:2411-8; Sun, et al. (2016) Molecules 21(8):1067; Khan, et al. (2017) Sci. Rep. 7:43789).

Flavone glycosides and flavone aglycones can be optionally isolated from the Scutellaria extract by precipitation with zinc acetate, the pH of which as been adjusted with at least about 14.7 mM ammonium hydroxide (Sun, et al. (2016) Molecules 21(8):1067). Alternatively, baicalin and wogonoside can be purified from a crude extract of S. baicalensis using ethyl acetate/water/1-n-oxtyl-3-methylimidazolium hexafluorophosphate ([C₄mim][PF₆]) (5:5:0.2, v/v) as a two-phase solvent system with purities of 99.3% and 99.1%, respectively, being obtained (Wang, et al. (2013) J. Liquid Chromatography Rel. Technol. 37(16):2275-86). Further, resin adsorption may be used to separate and purify baicalin and wogonoside from Scutellaria extracts. For example, after one round treatment with HPD-100 resin, recovery yields of 85.7% and 65.6%, are respectively obtained for baicalin and wogonoside (Du, et al. (2012) J. Chromatogr. B Analyt. Technol. Biomed. Life Sci. 908:143-9).

Other suitable methods for isolating flavones can include chromatographic fractionation based on molecular sizing, charge, solubility and/or polarity. Depending on the type of chromatographic method, column chromatography can be carried out with matrix materials composed of, for example, dextran, agarose, polyacrylamide or silica and can include solvents such as dimethyl sulfoxide, pyridine, water, dimethylformamide, methanol, saline, ethylene dichloride, chloroform, propanol, ethanol, isobutanol, formamide, methylene dichloride, butanol, acetonitrile, isopropanol, tetrahydrofuran, dioxane, chloroform/dichloromethane, etc. Typically, the product of the chromatographic step is collected in multiple fractions, which may then be tested for the presence of the desired compound using any suitable analytical technique (e.g., thin layer chromatography, mass spectrometry). Fractions enriched in the desired flavones may then be selected for further purification. In certain embodiments, an isolated flavone is at least 50%, 60%, 70%, 80%, 90%, 95%, or 99% pure.

Alternatively, the flavones of this invention may be chemically synthesized. For example, baicalein may be synthesized via Helilandin B (Chen, et al. (2010) J. Asian Nat. Prod. Res. 12(2):124-8) with subsequent Koenigs-Knorr glycosylation and mild-basic deprotection to produce baicalin (Li, et al. (2015) Tetrahedron Lett. 56(24):3816-9). Likewise, wogonin may be synthesized using 2,4-dibenzyloxy-6-hydroxy phenylacetone and benzaldehyde as the starting materials (Yuan, et al. (2016) Chin. J. Organ. Chem. 36(12):2960) with subsequent glycosylation to produce wogonoside.

As a further alternative, the flavones of this invention may be produced by recombinant means. By way of illustration, baicalein may be produced in recombinant E. coli cells from available phenylalanine and tyrosine (Jianhua, et al. (2019) Metab. Eng. 52:124-133). Further, selective C6-hydroxylation of 5,7-dihydroxyflavone using whole yeast cells stably expressing human CYP1A1 enzyme has been used to produce baicalein from chrysin (Ibidapo, et al. (2017) J. Agric. Food Chem. 65(34):7440-46).

When a flavone glycoside-enriched extract of Scutellaria or isolated flavone glycoside of the same is subjected to treatment with one or more carbohydrases, flavone glycosides are converted to flavone aglycones, which exhibit enhanced taste modulating activity. Accordingly, this invention also provides a consumable containing a carbohydrase-treated Scutellaria extract, or one or more flavone aglycones thereof. In some embodiments, the carbohydrase-treated Scutellaria extract is a carbohydrase-treated S. baicalensis extract. In particular embodiments, the carbohydrase-treated Scutellaria extract is enriched for one or more flavone aglycones, in particular flavone aglycones.

The carbohydrase-treated Scutellaria extract and/or flavone aglycones of the same may be produced by treating a Scutellaria extract or Scutellaria plant material (e.g., dry root powder) or flavone glycosides with one or more carbohydrases. As used herein, a “carbohydrase” refers to any enzyme that hydrolyses carbohydrates into simple sugars. Because carbohydrases act as a catalyst for the hydrolytic breakdown of the carbohydrate bonds into smaller units such as glucose or sucrose in the presence of water, they are considered hydrolases. In some embodiments, the carbohydrase is one or a combination of carbohydrases, i.e., a mixture of carbohydrase enzymes. The carbohydrase or carbohydrase mixture typically is selected from the group of saccharidase, amylase, exo-amylase, beta-amylase, gluco-amylase, endoamylase, alpha-amylase, glucanase, arabanase, hemicellulase, xylanase, and cellulase. In certain embodiments, the carbohydrase has endo-beta-glucanase activity that hydrolyzes (1,3)- or 1,4-linkages. In some embodiments, the carbohydrase or carbohydrase mixture includes at least a cellulase (e.g., XW-G-F Cellulase available from Novozyme, Denmark), which has been shown to yield wogonin and baicalein from wogonoside and baicalin, respectively (Yu, et al. (2013) Oncol. Rep. 30:2411-8). A recombinant β-glucuronidase from Lactobacillus brevis has also been shown to completely transform baicalin and wogonoside into baicalein and wogonin, respectively, within 3 hours (Sung, et al. (2009) J. Microbiol. Biotechnol. 19(12):1650-5). See also KR 20100001908 A. An exemplary carbohydrase of use in this invention is sold under the trademark VISCOZYME® L (Novozymes, Denmark).

By adding a carbohydrase to a Scutellaria extract or flavone glycoside in the presence of water for a time and at a pH and temperature that is sufficient to increase the reducing sugars, a carbohydrase-treated Scutellaria extract and flavone aglycone is produced. In some embodiments, the Scutellaria extract or flavone glycoside (e.g., 1 part) is combined with a carbohydrase or carbohydrase mixture (e.g., 0.005 to 0.1 part) in the presence of water (e.g., 5 to 50 parts) and is incubated for a time of about 30 minutes to about 48 hours, more preferably about 2 hours to about 24 hours; at a pH in the range of about 3 to about 6, or more preferably in the range of about 3.3 to about 5.5; and at a temperature between about 25° C. and 55° C., or more preferably between about 30° C. and 50° C.

The Scutellaria extract and flavones described herein improve the taste and/or flavor of a consumable by masking the astringency, bitterness and/or off-taste of a consumable, which has a component that imparts said astringent, bitter and/or off-taste. In this respect, a consumable includes any food product, pharmaceutical composition, dietary supplement, nutraceutical, dental hygienic composition, tabletop sweetener, beverage, or cosmetic product that includes a component having an astringent, bitter, and/or off-flavor. Preferably, the consumable having a component with an astringent, bitter or off-taste is modified by adding (a) a Scutellaria extract, (b) a carbohydrase-treated Scutellaria extract, (c) a S. baicalensis extract, (d) a carbohydrase-treated S. baicalensis extract, (e) a Scutellaria root extract, (f) a carbohydrase-treated Scutellaria root extract, (g) a S. baicalensis root extract, (h) a carbohydrase-treated S. baicalensis root extract, (i) one or more flavones obtained from a Scutellaria extract or other suitable source, (j) one or more flavones obtained from a Scutellaria root extract, (k) one or more flavones obtained from a S. baicalensis extract, (l) one or more flavones obtained from a S. baicalensis root extract, (m) one or more flavone aglycones obtained from a Scutellaria extract or other suitable source, (n) one or more flavone aglycones obtained from a Scutellaria root extract, (o) one or more flavone aglycones obtained from a S. baicalensis extract, (p) one or more flavone aglycones obtained from a S. baicalensis root extract, (q) one or more flavone glycosides obtained from a Scutellaria extract or other suitable source, (r) one or more flavone glycosides obtained from a Scutellaria root extract, (s) one or more flavone glycosides obtained from a S. baicalensis extract, (t) one or more flavone glycosides obtained from a S. baicalensis root extract, (u) baicalein, wogonin, oroxylin A, or a combination thereof, (v) baicalin, wogonoside, oroxylin A glucuronide, or a combination thereof, (w) baicalein, wogonin, oroxylin A, baicalin, wogonoside, oroxylin A glucuronide, or a combination thereof, or (x) a S. baicalensis root extract or a carbohydrase-treated S. baicalensis root extract in combination with one or more of baicalein, wogonin, oroxylin A, baicalin, wogonoside, or oroxylin A glucuronide.

The term “mask” or “masking” as used herein, is defined as covering, disguising, and/or obscuring an astringent, bitter, and/or off-flavor by the addition of a Scutellaria extract and/or flavones, wherein the component associated with the astringent, bitter, and/or off-flavor remains unchanged, but its unpleasant taste is not perceived by a human consuming said consumable. The taste and/or flavor profile of a consumable including the Scutellaria extract and/or flavones of the invention may be improved or enhanced (e.g., by 1.5-, 2.0-, 2.5-, 5.0-, 7.5- or 10-fold improvement) compared to the taste and/or flavor profile of a comparative consumable which does not include the Scutellaria extract and/or flavones as exogenous additives. Ideally, the Scutellaria extract and/or flavone reduces the off-flavor taste by at least about 10%, 20%, 30%, 40%, 50%, 60%, 70%, 80%, 90%, or 95%, or from about 60% to about 99%, or alternatively from about 20% to about 50% compared to the consumable not including the Scutellaria extract and/or flavone.

In certain embodiments, the Scutellaria extract and/or flavones of the invention reduce, suppress or mask the astringency, bitterness, and/or off-flavor of a consumable. An “off-flavor” or “off-taste” refers to a bitter, sour, fishy, earthy, astringent, metallic and/or unpleasant taste of a consumable. “Astringent” or “astringency” refers to a puckering or mouth drying sensation felt in the oral cavity. “Bitter” or “bitterness” refers to one of the four basic tastes, perceived primarily at the back of the tongue, which is often described as sharp, pungent, or disagreeable.

The component having an astringent, bitter and/or off-taste can be a protein, carbohydrate sweetener, artificial sweetener or preservative that is inherently present in the consumable (e.g., in food products containing fruits) or said component is added to the consumable. A protein with an astringent, bitter, and/or off-flavor can include an amino acid, protein hydrolysate or protein component of a consumable, in particular a plant protein or milk of grass-eating animals. Sweeteners of the present invention include, but are not limited to, carbohydrate sweeteners such as sucrose, fructose, glucose, high fructose corn syrup (containing fructose and glucose), xylose, arabinose, rhamnose, and sugar alcohols, such as erythritol, xylitol, mannitol, sorbitol, or inositol. Artificial sweeteners include, but are not limited to, Natural Sweet Flavor #2 (WO 2012/129451), stevioside, rebaudioside A, rebaudioside B, rebaudioside C, rebaudioside D, rebaudioside E, rebaudioside F, dulcoside A, dulcoside B, stevia, alpha-glucosyl stevia, fructosyl stevia, galactosyl stevia, beta-glucosyl stevia, siamenoside, mogroside IV, mogroside V, Luo Han Guo sweetener, monatin and its salts, glycyrrhizic acid and its salts (e.g., as found in MAGNASWEET), curculin, thaumatin, monellin, mabinlin, brazzein, hernandulcin, phyllodulcin, glycyphyllin, phloridzin, trilobtain, baiyunoside, osladin, polypodoside A, pterocaryoside A, pterocaryoside B, mukurozioside, phlomisoside I, periandrin I, abrusoside A, cyclocarioside I, or a combination thereof. Examples of preservatives having an astringent, bitter and/or off-taste include, but are not limited to benzoic acid and sorbic acid.

When added to a consumable as an exogenous additive, a Scutellaria extract and/or flavone of the invention is used in an amount effective to reduce or suppress the astringent, bitter or off-taste of a component of the consumable having an astringent, bitter or off-taste. Ideally, the amount of Scutellaria extract and/or flavone included in the consumable does not impart any off-taste to the consumable. Preferably, the amount of Scutellaria extract and/or flavone present in the consumable is an amount as low as 0.01 ppm, an amount as low as 0.05 ppm, in an amount as low as 1 ppm, in an amount as low as 5 ppm, in an amount as low as 7.5 ppm, or in an amount as low as 10 ppm. The Scutellaria extract and/or flavone can be included in the consumable in an amount that is as high as 200 ppm, in an amount as high as 150 ppm, in an amount as high as 100 ppm or in an amount as high as 1000 ppm. The Scutellaria extract and/or flavone may further be present within any range delimited by any pair of the foregoing values, such as between 0.05 ppm and 150 ppm, between 0.1 ppm and 100 ppm, between 0.5 ppm and 50 ppm, between 0.5 ppm and 500 ppm for example. In particular embodiments, the Scutellaria extract and/or flavone is used within the range of 0.2 ppm to 2 ppm. The term “ppm” as used herein means part per million by weight or volume, for example, the weight of the component (in milligrams) per liter of solution, i.e., μg/ml.

Scutellaria extracts and flavones of this invention have been associated with a number of possible therapeutic benefits including, e.g., treatment of inflammation, fever, cough, dysentery, and hypertension. As a commercial supplement having a serving size of 250 mg, the recommended use of S. baicalensis root extract is 1-3 servings 1-2 times per day. In clinal trial studies, administration of three daily doses of S. lateriflora (350 mg) was not associated with any negative effects (Brock, et al. (2014) Phytother. Res. 28(5):692-8). Similarly, oral doses of baicalein in the range of 100-2800 mg have been shown to be safe and well-tolerated by healthy subjects (Li, et al. (2014) J. Ethnopharmacol. 156:210-5; Pang, et al. (2016) Clin. Drug Invest. 36(9):713-724). Given that the Scutellaria extract and/or flavone are used in amounts significantly lower than those suggested for achieving a therapeutic benefit, the instant compositions are distinct from the pharmaceuticals, dietary supplements and nutraceuticals described in the prior art. As such, the compositions of this invention may provide taste modulating activity without associated pharmacological activity.

The phrase “food product” as used herein includes, but is not limited to, fruits, vegetables, juices, meat products (e.g., ham, bacon and sausage), egg products, fruit concentrates, gelatins and gelatin-like products (e.g., jams, jellies, preserves, and the like) milk products (e.g., ice cream, sour cream and sherbet), icings, syrups including molasses, corn products, wheat products, rye products, soybean products, oat products, rice products and barley products, nut meats and nut products, cakes, cookies, confectionaries (e.g., candies, gums, fruit flavored drops, and chocolates), chewing gum, mints, creams, ice cream, pies and breads, and beverages such as coffee, tea, carbonated soft drinks (e.g., COKE® and PEPSI®), non-carbonated soft drinks, juices and other fruit drinks, sports drinks such as GATORADE®, alcoholic beverages, such as beers, wines and liquors, and KOOL-AID®. Food products also include condiments such as herbs, spices and seasonings, and flavor enhancers, such as monosodium glutamate. A food product also includes prepared packaged products, such as dietetic sweeteners, liquid sweeteners, granulated flavor mixes which upon reconstitution with water provide non-carbonated drinks, instant pudding mixes, instant coffee and tea, coffee whiteners, malted milk mixes, pet foods, livestock feed, tobacco, and materials for baking applications, such as powdered baking mixes for the preparation of breads, cookies, cakes, pancakes, donuts and the like. Food products also include diet or low-calorie food and beverages containing little or no sucrose. Especially preferred food products are carbonated beverages.

The consumable can also be a pharmaceutical composition. Preferred compositions are pharmaceutical compositions containing the Scutellaria extract and/or flavone and one or more pharmaceutically acceptable excipients. These pharmaceutical compositions can be used to formulate pharmaceutical drugs containing one or more active agents that exert a biological effect other than taste modulation. The pharmaceutical composition preferably further includes one or more active agents that exert a biological or pharmacological effect. Such active agents include pharmaceutical and biological agents that have an activity other than taste modulation. Such active agents are well known in the art. See, e.g., The Physician's Desk Reference. Such compositions can be prepared according to procedures known in the art, for example, as described in Remington's Pharmaceutical Sciences, Mack Publishing Co., Easton, Pa. In one embodiment, such an active agent includes bronchodilators, anorexiants, antihistamines, nutritional supplements, laxatives, analgesics, anesthetics, antacids, H₂-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof. In one embodiment, the active agent is a antipyretic or analgesic, e.g., ibuprofen, acetaminophen, or aspirin; laxative, e.g., phenolphthalein dioctyl sodium sulfosuccinate; appetite depressant, e.g., amphetamine, phenylpropanolamine, phenylpropanolamine hydrochloride, or caffeine; antacidic, e.g., calcium carbonate; antiasthmatic, e.g., theophylline; antidiuretic, e.g., diphenoxylate hydrochloride; agent active against flatulence, e.g., simethecon; migraine agent, e.g., ergotaminetartrate; psychopharmacological agent, e.g., haloperidol; spasmolytic or sedative, e.g., phenobarbitol; antihyperkinetic, e.g., methyldopa or methylphenidate; tranquilizer, e.g., a benzodiazepine, hydroxinmeprobramate or phenothiazine; antihistaminic, e.g., astemizol, chloropheniramine maleate, pyridamine maleate, doxlamine succinate, bromopheniramine maleate, phenyltoloxamine citrate, chlorocyclizine hydrochloride, pheniramine maleate, or phenindamine tartrate; decongestant, e.g., phenylpropanolamine hydrochloride, phenylephrine hydrochloride, pseudoephedrine hydrochloride, pseudoephedrine sulfate, phenylpropanolamine bitartrate, or ephedrine; beta-receptor blocker, e.g., propanolol; agent for alcohol withdrawal, e.g., disulfuram; antitussive, e.g., benzocaine, dextromethorphan, dextromethorphan hydrobromide, noscapine, carbetapentane citrate, or chlophedianol hydrochloride; fluorine supplement, e.g., sodium fluoride; local antibiotic, e.g., tetracycline or cleocine; corticosteroid supplement, e.g., prednisone or prednisolone; agent against goiter formation, e.g., colchicine or allopurinol; antiepileptic, e.g., phenyloine sodium; agent against dehydration, e.g., electrolyte supplement; antiseptic, e.g., cetylpyridinium chloride; NSAID, e.g., acetaminophen, ibuprofen, naproxen, or salt thereof; gastrointestinal active agent, e.g., loperamide and famotidine; an alkaloid, e.g., codeine phosphate, codeine sulfate, or morphine; supplement for a trace element, e.g., sodium chloride, zinc chloride, calcium carbonate, magnesium oxide, or other alkali metal salt or alkali earth metal salt; vitamin; ion-exchange resin, e.g., cholestyramine; cholesterol-depressant or lipid-lowering substance; antiarrhythmic, e.g., N-acetylprocainamide; or expectorant, e.g., guaifenesin.

In some embodiments, the consumable is a dietary supplement or nutraceutical. Examples of such compositions having an undesirable taste include, but are not limited to, enteral nutrition products for treatment of nutritional deficit, trauma, surgery, Crohn's disease, renal disease, hypertension, obesity and the like, to promote athletic performance, muscle enhancement or general well-being or inborn errors of metabolism such as phenylketonuria. In particular, such compositions can contain one or more amino acids which have a bitter or metallic taste or aftertaste. Such amino acids include, but are not limited to, essential amino acids such as L isomers of leucine, isoleucine, histidine, lysine, methionine, phenylalanine, threonine, tryptophan, tyrosine, and valine.

In a further embodiment, the consumable of the present invention is a dental hygienic composition, containing a Scutellaria extract and/or flavone of this invention. Dental hygienic compositions are known in the art and include, but are not necessarily limited to, toothpaste, mouthwash, plaque rinse, dental floss, dental pain relievers (such as ANBESOL™), and the like. In one embodiment, the dental hygienic composition includes one sweetener. In another embodiment, the dental hygienic composition includes more than one sweetener. In certain embodiments, the dental hygienic composition includes sucrose and corn syrup, or sucrose and aspartame.

In yet another embodiment, the consumable of the present invention is a cosmetic product containing a Scutellaria extract and/or flavone of this invention. For example, but not by way of limitation, the cosmetic product can be a face cream, lipstick, lip gloss, and the like. Other suitable compositions of the invention include lip balm, such as those sold under the trademarks CHAPSTICK® or BURT'S BEESWAX® Lip Balm.

The invention is described in greater detail by the following non-limiting examples.

### Example 1: Scutellaria Baicalensis Extracts

Scutellaria baicalensis Extract. Skullcap extract was obtained by drying and milling the root of Scutellaria baicalensis. To the dried powder was added water and ethanol (30:70). The resulting mixture was incubated for a time sufficient to extract the desired flavones and the mixture was filtered to remove insoluble plant material. The filtered solution was subsequently dried in a spray dryer and milled.

Carbohydrase-Treated S. baicalensis Extract. One part dried S. baicalensis extract was resuspended in 5 to 50 parts water. The pH of the solution was adjusted to 3-6 and 0.005 to 0.1 part carbohydrase sold under the trademark VISCOZYME® L (Novozymes, Denmark) was added. The mixture was incubated at 30-55° C. for 2-48 hours and subsequently cooled to room temperature. The solution was then concentrated by removing the water. In some instances, the concentrated sample was further purified to obtain the desired flavones by column chromatography.

Both the carbohydrase-treated and untreated S. baicalensis extract were analyzed do assess the concentration of flavones present in the extracts. The results of this analysis are presented in Table 2.

TABLE 2

| | | Carbohydrase-Treated |
|---|---|---|
| | S. baicalensis | S. baicalensis |
| Component | Extract (%) | Extract (%) |
| Baicalin | 28.4 | 6.62 |
| Oroxylin A | 1.4 | Low |
| glucuronide | | |
| Wogonoside | 4.1 | Low |
| Baicalein | 2.5 | 47.01 |
| Oroxylin A | 0.7 | 4.11 |
| Wogonin | NQ | 10.68 |

NQ, Not quantified due to the low concentration.

This analysis indicated that untreated S. baicalensis extract had a high content of baicalin and wogonoside. With bio-transformation, most baicalin was converted to baicalein; and wogonoside was converted to wogonin. The activity of the individual compounds was also analyzed for taste modulating activity. This analysis indicated that baicalin and wogonoside exhibited some activity, but baicalein and wogonin were more active.

### Example 2: Taste Modulation

A taste modulating composition composed of 43% baicalein and 11% wogonin was evaluated using different amounts of the composition in different applications. The results of this analysis are presented in Table 3.

TABLE 3

| | Taste | |
|---|---|---|
| | Modulating | |
| Base | Composition | Functions |
| Benzoic acid, | 0.5 to 1 ppm | Masked astringency, drying, |
| 250 ppm | | lingering and bitterness |
| Sorbic acid, | 0.5 to 1 ppm | Masked astringency, drying, |
| 150 ppm | | lingering and bitterness |
| Rebaudioside A, | 1 to 2 ppm | Reduced the drying and |
| 100 ppm | | lingering off-flavors and |
| | | provided nice sweet mouthfeel |
| Naringenin, | 1 to 2 ppm | Reduced the off-flavor and |
| 50 ppm | | makes the sample cleaner |
| Luo Han | 1 to 2 ppm | Reduced the off-flavor of Luo |
| extract, | | Han extract |
| 50 ppm | | |
| AquaBall | 2 to 4 ppm | Reduced the off-flavor |
| (flavored water | | (drying, lingering etc.) of |
| sweetened with | | the sample and provided |
| stevia) | | better sweet mouthfeel |
| Ciel Exprim | 2 to 4 ppm | Reduced the off-flavor |
| (flavored | | (drying, lingering, etc.) |
| water) | | |
| Pea protein, | 0.1 ppm | Reduced brown pea off-flavor, |
| 1% (Roquette) | | lingering and astringency |
| 800 ppm tannic | 1 ppm | Masked astringency, drying, |
| acid, 800 ppm | | lingering and bitterness. |
| tartaric acid, | | Brought in good mouthfeel |
| 800 ppm | | |
| phosphoric acid | | |
| (in water) | | |
| 1000 ppm malic | 1 ppm* | Masked the sharp sourness, |
| acid, 2000 ppm | | added mouthfeel |
| citric acid (in | | |
| water) | | |

*Taste modulating composition was composed only of wogonin.

What is claimed is:

1. A consumable comprising a component having an astringent, bitter or off-taste; and a Scutellaria extract or one or more flavones thereof.

2. The consumable of claim 1, wherein the component having an astringent, bitter or off-taste is a protein, carbohydrate sweetener, artificial sweetener or preservative.

3. The consumable of claim 1, wherein the Scutellaria extract is a carbohydrate-treated Scutellaria extract.

4. The consumable of claim 1, wherein the one or more flavones comprise flavone aglycones, flavone glycosides, or a combination thereof.

5. The consumable of claim 4, wherein the one or more flavone aglycones are baicalein, wogonin, oroxylin A, or a combination thereof.

6. The consumable of claim 4, wherein the one or more flavone glycosides are baicalin, wogonoside, oroxylin A glucuronide, or a combination thereof.

7. The consumable of claim 1, wherein the Scutellaria extract is a Scutellaria baicalensis extract.

8. The consumable of claim 1, wherein the consumable is a food product, pharmaceutical composition, a dietary supplement, a nutraceutical, a dental hygienic composition, a tabletop sweetener, a beverage, or a cosmetic product.

9. The consumable of claim 1, wherein the Scutellaria extract or one or more flavones thereof is present in an amount of 0.01 ppm or greater.

10. The consumable of claim 1, wherein the Scutellaria extract or one or more flavones thereof is present in an amount in the range of 0.05 ppm to 500 ppm.

11. The consumable of claim 1, wherein the Scutellaria extract or one or more flavones thereof is present in an amount in the range of 0.1 ppm to 100 ppm.

12. The consumable of claim 1, wherein the Scutellaria extract or one or more flavones thereof is present in an amount in the range of 0.5 ppm to 50 ppm.

13. The consumable of claim 1, wherein the Scutellaria extract is a Scutellaria baicalensis extract.

14. A method of improving the taste of a consumable comprising adding to a consumable having a component with an astringent, bitter or off-taste, a Scutellaria extract or one or more flavones thereof in an amount effective to reduce or suppress said astringent, bitter or off-taste thereby improving the taste of a consumable.

15. The method of claim 14, wherein the component with an astringent, bitter or off-taste is a protein, carbohydrate sweetener, artificial sweetener or preservative.

16. The method of claim 14, wherein the Scutellaria extract is a carbohydrase-treated Scutellaria extract.

17. The method of claim 14, wherein the one or more flavones comprise flavone aglycones, flavone glycosides, or a combination thereof.

18. The method of claim 17, wherein the one or more flavone aglycones are baicalein, wogonin, oroxylin A, or a combination thereof.

19. The method of claim 17, wherein the one or more flavone glycosides are baicalin, wogonoside, oroxylin A glucuronide, or a combination thereof.

20. The method of claim 14, wherein the consumable is a food product, pharmaceutical composition, a dietary supplement, a nutraceutical, a dental hygienic composition, a tabletop sweetener, a beverage, or a cosmetic product.
